(19) United States
(12) Patent Application Publication
(10) Pub. No.: US 2018/0311376 A1
(43) Pub. Date: Nov. 1, 2018

(54) A Medical Composition and a Medical Hydrogel for Use in the Prevention and/or Treatment of a Disease of the Facet Joints and/or for Use in the Replacement and/or Regeneration of Articular Facets

(71) Applicant: TETEC Tissue Engineering Technologies AG, Reutlingen (DE)

(72) Inventors: Helmut Wurst, Kusterdingen (DE); Christoph Gaissmaier, Kusterdingen (DE); Nils Clausen, Tübingen (DE)

(21) Appl. No.: 15/770,411

(22) PCT Filed: Oct. 20, 2016
(86) PCT No.: PCT/EP2016/075233

§ 371 Date: Apr. 23, 2018

(30) Foreign Application Priority Data
Oct. 21, 2015 (DE) 10 2015 220 583.5

(51) Int. Cl. A61K 47/69; A61K 38/38; A61K 47/10; A61K 47/42; A61P 19/02; A61K 9/00; A61K 31/728

(52) U.S. Cl. CPC A61K 47/6903; A61K 38/385; A61K 47/10; A61K 47/42; A61K 31/16; A61K 9/0019; A61K 31/728; A61K 2300/00; A61P 19/02

(57) ABSTRACT

The invention relates to medical compositions, a medical hydrogel, a medical kit and a multichamber discharge device for use in the prevention and/or treatment of a disease of the facet joints and/or in the replacement and/or regeneration of articular facets.

### FIELD OF APPLICATION AND PRIOR ART

The invention relates to medical compositions, a medical hydrogel and a medical kit for use in the prevention and/or treatment, in particular symptomatic treatment, of a disease of the facet joints and/or in the replacement and/or regeneration of articular facets.

The spine undergoes wear during the normal aging process or due to excessive loads. In the course of these changes, the so-called facet joints (intervertebral joints) are also subjected to heavy loads.

The facet joints are small, planar paired sets of joints that connect the dorsal processes of adjacent vertebrae with one another, thus ensuring spinal mobility. The articular surfaces comprise a cartilaginous sliding layer via which movement takes place. Each of the joints is surrounded by an articular capsule, into which grow not only the vessels necessary for blood supply but also nerves for the perception of pain.

Like every joint, the facet joint also undergoes degenerative changes over the course of a lifetime. In normal cases, the facet joints are only subjected to a minor pressure load. The most frequent cause of degenerative changes in the facet joints is a reduction in the height of the disk space resulting from disk wear, herniated disks, or surgical removal of a disk. Even a decrease in the height of the disk space of a few millimeters increases the pressure load on the facet joints many times over. The elevated pressure load results in increasing cartilage abrasion. The increasing cartilage abrasion is accompanied by gradual loss of competence of the cartilage structure, which finally manifests itself as clinically apparent arthrosis.

Facet joint disease is perceived by the patient in the form of severe back pain. The first pain ordinarily occurs immediately after getting up in the morning when the spinal column must again support the body weight and is thus somewhat compressed. The worn areas of the cartilage slide over one another, resulting in pain. An aggravating factor is that the damaged joints show so-called osteophytic reactions. This means that because of the progressive degeneration, the facet joints tend to form and build up new but superfluous bone. This can lead to (additional) stenosis of the spinal foramina, so that the pressure on the stenosed nerves causes the symptom of radiating pain. In facet joint disease, therefore, leg pain may occur in addition to the typical back pain.

Therapy for facet joint disease essentially encompasses the entire spectrum of conservative treatment options, including physical therapy. In addition, causal therapy should be provided whenever possible. In the majority of cases, however, this is not possible because of the advanced degenerative changes such as disk wear at other sites, but arthrosis of the facet joints no longer allows freedom from pain. Symptomatic treatment is indicated in such cases.

An example of a symptomatic treatment approach is so-called facet joint infiltration (abbreviated as facet infiltration). This is a type of microinvasive pain therapy in which locally acting pharmaceutical substances are injected into the facet joints. The objective is pain reduction or alleviation of inflammation. The procedure is carried out using imaging techniques (such as computed or magnetic resonance tomography), as blind injection would miss the facet joints, and in particular their articular capsules. As a rule, a local anesthetic is injected in combination with an anti-inflammatory such as a cortisone preparation.

Moreover, hyaluronic acid preparations, with have a chondroprotective action, have now come into use in facet joint infiltration as well.

However, a disadvantage of this procedure is that the injected drugs or active ingredients rapidly diffuse out of the treated facet joints, and depending on the drug or active ingredient used—for example in the case of hyaluronic acid—are relatively quickly resorbed. The overall result is rapid loss of the active ingredient in the facet joints. As a rule, therefore, it is only possible to alleviate the patient's symptoms on a temporary basis. In most cases, multiple injections are required in order to achieve a clinically significant reduction in symptoms.

### OBJECT AND ACHIEVEMENT THEREOF

The object of the invention is therefore to provide a medical composition, a medical hydrogel and a medical kit that allow improved therapy, i.e., prevention and/or treatment, of facet joint disease and defects compared to the prior art.

This object is achieved by means of a medical composition according to claim 1 and a medical hydrogel according to claim 18. Preferred embodiments are defined in the dependent claims. The wording of all of the claims is hereby incorporated into the description by express reference.

In addition, further embodiments by means of which the object of the invention is achieved are disclosed in the description.

According to a first aspect, the invention relates to a medical composition, i.e. a composition usable in the field of medicine, for use in the prevention (prophylaxis) and/or treatment, in particular symptomatic treatment, of disease of the facet joints (facet joint disease), in particular in humans or animals, and/or a medical composition for use in the replacement and/or regeneration of articular facets, in particular human or animal articular facets.

In particular, the composition is characterized by comprising a crosslinkable protein.

The composition, in particular the protein, can preferably be converted into a hydrogel-forming material or a hydrogel by means of a crosslinking agent, in particular a chemical crosslinking agent. In other words, the composition, in particular the protein, is preferably characterized in that said composition, in particular the protein, can be crosslinked into a hydrogel-forming material or hydrogel.

Within the meaning of the present invention, the term “facet joints” is to be understood as referring to at least one facet joint, i.e. one facet joint or a plurality of facet joints, such as e.g. two, three, four, etc., facet joints.

Within the meaning of the present invention, the term “disease of the facet joints” or “facet joint disease” is to be understood as referring to a physical, in particular functional and/or morphological change in the facet joints that constitutes deterioration compared to full physiological capacity (of a healthy human and/or a healthy animal).

Within the meaning of the present invention, the term “animal” refers to animals, preferably vertebrates, other than humans. Accordingly, for example, the term “animal articular facets” refers within the meaning of the present invention to all animal articular facets, but excludes human articular facets.

Within the meaning of the present invention, the term “crosslinkable protein” is to be understood as referring to a protein that can be crosslinked using a crosslinking agent, in particular a chemical crosslinking agent, because of its chemical properties, in particular because of suitable functionalization or derivatization. The crosslinking can take place in particular by means of hydrogen bonds, ionic bonds, and/or covalent bonds between the protein and the crosslinking agent. The crosslinking of the protein preferably takes place by means of covalent bonds between the protein and the crosslinking agent.

Within the meaning of the present invention, the term “crosslinking agent” is to be understood as referring to a compound comprising at least two reactive groups, in particular only two reactive groups. As will be discussed in further detail below, the reactive groups are preferably thiol groups, i.e. SH groups.

Within the meaning of the present invention, the term “hydrogel” is to be understood as referring to a water-containing, three-dimensional polymer network (or a water-containing, three-dimensional polymer matrix).

Production of the network (or the matrix) preferably results from the formation of chemical and/or physical bonds or links between starting components (such as in particular a crosslinkable protein and a crosslinking that are used to produce the network (or matrix). The chemical bonds may be covalent and/or non-covalent bonds. The bonds are preferably covalent bonds, at least predominantly. Non-covalent bonds may be selected from the group comprising ionic bonds, hydrogen bonds, dipoledipole interactions, Van der Waals forces, and combinations of two or more of the above-mentioned non-covalent bonds.

The invention is based on the surprising finding that such a crosslinkable protein can be crosslinked in the presence of active ingredients, such as e.g. chondroprotective agents, anti-inflammatories, local anesthetics or the like, into a hydrogel-forming material or hydrogel, resulting in delayed (prolonged) release of the active ingredients. When such a hydrogel-forming material or hydrogel is produced in diseased facet joints or administered into diseased facet joints, the active ingredient loss (due to diffusion and in particular resorption) known from the prior art can be slowed. Because of the delayed-release effect described above, the active ingredients contained in the hydrogel-forming material or the hydrogel remain in the facet joints locally for a longer period of time, in particular in their articular capsules or at least in the vicinity of the facet joints. This allows the active ingredients to exert their desired action over a longer period of time. This makes it possible to provide treatment of facet joint disease that is significantly more effective and longer-lasting than generic therapeutic approaches. At the same time, the hydrogel-forming material or hydrogel can serve as a replacement structure for diseased articular facets and/or as a structure for reconstruction, i.e. neogenesis, of the articular facets.

Moreover, the longer presence of the active ingredient in the facet joints makes it possible to sharply reduce the number of treatment cycles required to achieve noticeable therapeutic success, for example the number of injections in facet joint infiltration. On the one hand, this improves the quality of life for the patient. On the other, it makes it possible to reduce treatment costs to a considerable extent.

A further advantage is that the hydrogel-forming material or hydrogel can be characterized by antineurotrophic, antiosteogenic and/or antiadhesive properties, making it possible to further improve the possibilities for prevention and/or treatment of facet joint disease and replacement and/or regeneration of articular facets.

A further advantage is that the hydrogel-forming material or hydrogel acts as an infiltration barrier to inflammatory cells and thus exerts an anti-inflammatory effect.

Finally, it is advantageous that the composition, in particular the protein, can be readily combined with active ingredients, which will be discussed in further detail below.

It is true that crosslinkable proteins were already generally known from DE 10 2008 008 071 A1 and DE 10 2009 051 575 A1 in connection with the treatment of cartilage and disk defects and arthritis. However, their use for the prevention and/or treatment, in particular symptomatic treatment, of facet joint diseases and for replacement and/or regeneration of articular facets was not known.

In a preferred embodiment, the formation of the hydrogel-forming material or hydrogel is based on a Michael addition between the protein and the crosslinking agent, wherein the protein preferably reacts as a Michael acceptor and the crosslinking agent as a Michael donor. In a further embodiment, the facet joints are lumbar, thoracic and/or cervical facet joints. The facet joints are preferably lumbar facet joints.

In a further embodiment, the articular facets are lumbar, thoracic and/or cervical articular facets. The articular facets are preferably lumbar articular facets.

The term “lumbar” means “pertaining to the lumbar region.” Within the meaning of the present invention, the term “thoracic” means “pertaining to the chest area (thorax).” Within the meaning of the present invention, the term “cervical” means “pertaining to the neck.”

In a further embodiment, the disease of the facet joints is a degenerative disease of the facet joints, i.e. a degenerative facet joint disease.

Within the meaning of the present invention, the term “degenerative disease of the facet joints” or “degenerative facet joint disease” is to be understood to refer to facet joint disease that is based on a functional and/or morphological change in the facet joints that is a change for the worse compared to full physiological capacity (of a healthy human and/or animal). Within the meaning of the present invention, the causes of degenerative facet joint disease may in particular be advanced age, excess load (for example, elevated body weight), congenital or traumatic causes such as malposition of the facet joints, bony deformation caused by bone diseases (for example, osteoporosis and/or other diseases such as arthritis) or (prior) injuries of the facet joints. Degenerative facet joint disease within the meaning of the present invention is characterized in particular by loss of competence of the facet joints, in particular the affected articular facets.

In a particularly preferred embodiment, the disease, in particular the degenerative disease, of the facet joints is arthrosis of the facet joints, i.e. facet joint arthrosis.

In general, the arthrosis can be a primary or idiopathic arthrosis, a secondary or symptomatic arthrosis, or an activated arthrosis.

Within the meaning of the present invention, the term “primary arthrosis or idiopathic arthrosis” is to be understood as referring to arthrosis caused by an imbalance between the load and the load-bearing capacity of the articular facet (overweight, overloading etc.), congenital malposition or articular dysplasias. Within the meaning of the present invention, the term “secondary or symptomatic arthrosis” is to be understood as referring to arthrosis of traumatic (post-traumatic joint malposition, post-traumatic joint injuries, etc.), metabolic (tissue or cartilage damage due to ischemia, diabetes mellitus, alcoholism, inflammation, etc.) or pharmaceutical (for example due to gyrase inhibitors) origin.

Within the meaning of the present invention, the term “activated arthrosis” is to be understood as referring to arthrosis accompanied by inflammation (“inflamed” arthrosis).

According to a particularly preferred embodiment, the disease of the facet joints is secondary or symptomatic arthrosis of the facet joints.

In a further embodiment, the disease of the facet joints is an inflammatory disease of the facet joints, i.e. inflammatory facet joint disease.

The disease, in particular the inflammatory disease, of the facet joints is preferably arthritis of the facet joints, i.e. facet joint arthritis.

In general, the composition can be designed to be converted into the hydrogel-forming material or hydrogel during or after administration thereof.

In particular, the composition can be configured for in situ conversion into the hydrogel-forming material or hydrogel. In other words, it can be provided in particular according to the invention that the composition is not converted into the hydrogel-forming material or hydrogel until it is in situ, i.e. directly at the site to be treated.

In a further embodiment, the composition is in sprayable form.

The composition is preferably in injectable form.

In general, the composition can be configured for surgical administration. In other words, the composition can be surgically used or applied.

According to a preferred embodiment, however, the composition is configured for microinvasive administration. In other words, however, it is preferred according to the invention if the composition is microinvasively used or applied.

According to a particularly preferred embodiment, the composition is configured for facet joint infiltration, i.e. for infiltration, preferably injection, into the facet joints. In other words, the composition is particularly preferably used for facet joint infiltration, i.e. for infiltration, preferably injection, into the facet joints or administered, preferably injected, into the facet joints.

It is particularly preferable if the composition is configured for intra-articular facet joint infiltration, i.e. for infiltration, preferably injection, into the articular capsules of the facet joints. In other words, it is particularly preferred if the composition is used for intra-articular facet joint infiltration, i.e. for infiltration, preferably injection, into the articular capsules of the facet joints or administered, preferably injected, into the articular capsules of the facet joints.

Alternatively or in combination, the composition can be configured for periarticular facet joint infiltration, i.e. for infiltration, preferably injection, into the vicinity of the facet joints or around them (outside of the articular capsules). In other words, it can be alternatively or additionally provided according to the invention that the composition be used for periarticular facet joint infiltration or administered, preferably injected, into the vicinity of the facet joints or around them (outside of the articular capsules).

In a further embodiment, the medical composition is an acellular composition, i.e. a composition that is free of cells.

In a further embodiment, the protein provided according to the invention is a hydrophilic protein, in particular a water-soluble protein.

In further embodiments, the protein is a naturally occurring protein, in particular a blood serum protein.

In an alternative embodiment, the protein is an artificially produced protein. For example, the protein can be a chemically synthesized or recombinantly produced protein.

In general, the protein can be of human or animal origin. For example, it may be a protein of bovine (cow) origin, porcine (pig) origin, equine (horse) origin, ovine (sheep) origin, caprine (goat) origin, or leporid (rabbit) origin. According to the invention, however, the protein should preferably be a human protein.

In general, moreover, the protein can be of allogeneic, autologous or xenogeneic origin. According to the invention, the protein should preferably be an allogeneic protein.

In a preferred embodiment, the protein is a functionalized protein.

Within the meaning of the present invention, the term “functionalized protein” is to be understood as referring to a protein which, for example by means of the addition of functional groups, possesses a functional group it ordinarily does not have.

In a preferred embodiment, the protein is functionalized by functional groups, in particular thiol-reactive groups.

Within the meaning of the present invention, the term “thiol-reactive groups” is to be understood as referring to functional groups that are capable of reacting with thiol and/or thiolate groups of another compound, preferably a crosslinking agent.

In a further embodiment, the functional groups can be selected from the group comprising or composed of maleimide groups, vinyl sulfone groups, acrylate groups, alkyl halide groups, azirine groups, pyridyl groups, thio-nitrobenzoic acid groups, arylating groups and combinations of two or more of the above-mentioned functional groups.

The protein is preferably a maleimide-functionalized protein, in particular a maleimide-functionalized serum protein, preferably of human origin.

In a further embodiment, the thiol groups, preferably all of the thiol groups, of the protein are present in blocked form. The blocking of the thiol groups is preferably carried out by reaction of the protein with a maleimide compound, in particular N-maleoyl-β-alanine (3-maleimidopropionic acid) or 3-maleimidopropionic acid-N-hydroxysuccinimide ester.

In a further embodiment, primary amino groups of the protein are present in blocked form. A significant percentage of the primary amino groups, in particular 10% to 70% of the primary amino groups, is preferably present in blocked form. The blocking is preferably carried out by reaction of the protein with a maleimide-functionalized active ester, in particular 3-maleimidopropionic acid-N-hydroxysuccinimide ester.

Within the meaning of the present invention, the term “active ester” is to be understood as referring to an ester that has increased acylation potential because of a particularly pronounced electron-attracting action of its alcohol portion.

In a further embodiment, the protein is produced or producible from a refunctionalized protein by reaction with N-maleoyl-β-alanine and subsequent reaction with 3-maleimidopropionic acid-N-hydroxysuccinimide ester.

In a further embodiment, the protein is contained in an amount of 0.3 wt % to 20 wt %, in particular 0.4 wt % to 5 wt %, preferably 0.5 wt % to 2 wt %, based on the total weight of the composition.

In a further embodiment, the protein is selected from the group comprising or composed of crosslinkable albumin, crosslinkable serum proteins, derivatives thereof and mixtures of two or more of the above-mentioned proteins.

Within the meaning of the present invention, the term “derivative” or “derivatives” is to be understood as referring to a protein that is produced by derivatization from an unfunctionalized protein. Examples of derivatization processes include covalent binding of functional groups and/or biological factors, modification of the charge and/or polarization status and/or modification of the protein structure.

In a particularly preferred embodiment, the protein is a maleimide-functionalized albumin, in particular maleimide-functionalized serum albumin, preferably of human origin.

In a further embodiment, the composition further comprises at least one active ingredient.

The active ingredient is preferably selected from the group comprising or composed of a chondroprotective agent, an anti-inflammatory, an analgesic, an antibiotic, a local anesthetic, a cytostatic, a differentiation factor, a modulation factor, an immunosuppressant, an immunostimulant active ingredient, an apoptosis-inducing active ingredient and mixtures of two or more of the above-mentioned active ingredients.

Within the meaning of the present invention, the term “chondroprotective agent” is to be understood as referring to a cartilage-protecting compound. The chondroprotective action results as a rule from inhibition of cartilage-degrading substances and thus reduction of loss of protective cartilage. At the same time, the regeneration of cartilage tissue is generally promoted.

In particular, the chondroprotective agent can be selected from the group comprising or composed of hyaluronic acid, chondroitin sulfate, glucosamine sulfate, oxaceprol, antioxidants (such as vitamin E), ademetionine (S-adenosylmethionine, SAM), derivatives of the above-mentioned chondroprotective agents and mixtures of two or more of the above-mentioned chondroprotective agents.

The chondroprotective agent is preferably hyaluronic acid. The hyaluronic acid can be either non-crosslinked or crosslinked.

In a further embodiment, the chondroprotective agent is contained in an amount of 0.1 wt % to 5 wt %, in particular 0.2 wt % to 2 wt %, preferably 0.3 wt % to 1 wt %, based on the total weight of the composition.

In a further embodiment, the medical composition further comprises a local anesthetic, i.e. a locally acting anesthetic, selected from the group comprising or composed of lidocaine, mepivacaine, prilocaine, articaine, bupivacaine, ropivacaine, etidocaine, dyclonine, procaine, benzocaine, 2-chloroprocaine, oxybuprocaine, tetracaine, fomocaine, derivatives of the above-mentioned local anesthetics and mixtures of two or more of the above-mentioned local anesthetics.

In a further embodiment, the local anesthetic is contained in an amount of 0.05 wt % to 15 wt %, in particular 0.1 wt % to 10 wt %, preferably 0.2 wt % to 5 wt %, based on the total weight of the composition.

The above-mentioned anti-inflammatory (antiphlogistic or inflammation inhibitor) can be selected from the group comprising or composed of a steroidal anti-inflammatory, a nonsteroidal anti-inflammatory, a vegetable anti-inflammatory and mixtures of two or more of the above-mentioned anti-inflammatories.

For example, the anti-inflammatory can be selected from the group comprising or composed of ibuprofen, acetylsalicylic acid, diclofenac, indomethacin, phenylbutazone, dexamethasone, hydrocortisone, prednisolone, prednisone, betamethasone, triamcinolone, essential oils or extracts, particularly from camomile and/or arnica blossoms, derivatives of the above-mentioned anti-inflammatories (such as betamethasone acetate, triamcinolone acetonide, triamcinolone hexacetonide and/or methylprednisolone) and mixtures of two or more of the above-mentioned antiinflammatories.

In a further embodiment, the anti-inflammatory is contained in an amount of 0.02 wt % to 15 wt %, in particular 0.05 wt % to 10 wt %, preferably 0.1 wt % to 5 wt %, based on the total weight of the composition.

In a further embodiment, the composition further comprises a crosslinking agent. The crosslinking agent is preferably spatially separated from the crosslinkable protein. In this manner, premature crosslinking of the protein can be prevented. In other words, according to a further embodiment, the composition comprises a crosslinkable protein and a crosslinking agent that are spatially separated from each other. In this embodiment, the composition can in particular be in the form of a kit. With respect to the crosslinkable protein, the preceding description is incorporated herein by reference in its entirety.

In general, the crosslinking agent can be a homomultifunctional, in particular homobifunctional, or a heteromultifunctional, in particular heterobifunctional, agent. The crosslinking agent is preferably a homobifunctional crosslinking agent.

In a further embodiment, the crosslinking agent comprises thiol groups (SH groups).

In particular, the crosslinking agent can be a crosslinking agent functionalized or modified with thiol groups.

Moreover, the crosslinking agent can also be a polymer, in particular a polymer functionalized or modified with thiol groups.

For example, the crosslinking agent can be selected from the group comprising or composed of dithiopolyethylene glycol (dithio-PEG), SH-modified hyaluronic acid, SH-modified dextran, SH-modified polyvinyl alcohol, SH-modified polyvinylpyrrolidone and mixtures of two or more of the above-mentioned crosslinking agents.

According to a particularly preferred embodiment, the crosslinking agent is a polymer functionalized or modified with thiol groups, in particular dithio-polyethylene glycol (dithio-PEG).

In a particularly preferred embodiment, the crosslinkable protein is maleimide-functionalized albumin, in particular maleimide-functionalized serum albumin, and the crosslinking agent is dithio-polyethylene glycol (dithio-PEG).

In this case, the crosslinking reaction takes place by means of a Michael addition between the maleimide groups of the albumin (Michael acceptor) and the thiol groups of the dithio polyethylene glycol (Michael donor).

In a further embodiment, the composition further comprises salts and/or saccharides. In particular, the can be sodium chloride and/or buffer salts, preferably a mixture of sodium dihydrogen phosphate and disodium hydrogen phosphate. A particular example of the saccharides is trehalose.

In a further embodiment, the medical composition further comprises a contrast agent. The use of a contrast agent allows particularly advantageous imaging-based treatment, for example by means of computed or magnetic resonance tomography.

For example, the contrast agent can be selected from the group comprising or composed of iodinated or iodine-substituted contrast agents, barium sulfate and mixtures of two or more of the above-mentioned contrast agents.

In an advantageous embodiment from the standpoint of storage, the composition, in particular the crosslinkable protein and/or a crosslinking agent that is optionally additionally present, are in solid form, in particular as a lyophilizate.

In a further embodiment, the medical composition, in particular the crosslinkable protein and/or a crosslinking agent that is optionally additionally present, is present in the form of an aqueous liquid, in particular in the form of a suspension or solution, preferably a solution.

According to a second aspect, the invention relates to a medical-hydrogel-forming material or a medical hydrogel, i.e. a hydrogel-forming material or a hydrogel that is usable in the field of medicine, for use in the prevention (prophylaxis) and/or treatment, in particular symptomatic treatment, of a disease of the facet joints (facet joint disease), in particular in humans or animals, and/or a medical hydrogel-forming material or a hydrogel for use in the replacement and/or regeneration of articular facets, in particular human or animal articular facets.

The hydrogel-forming material or hydrogel is obtainable or producible by mixing of a crosslinkable protein and a crosslinking agent.

By means of the mixing, a crosslinking reaction takes place between the protein and the crosslinking agent, causing a hydrogel-forming material or hydrogel to be formed.

In particular, the hydrogel-forming material or hydrogel is obtainable or producible by mixing of an aqueous liquid, in particular an aqueous suspension or an aqueous solution, containing a crosslinkable protein, and an aqueous liquid, in particular an aqueous suspension or an aqueous solution, containing a crosslinking agent.

In general, the hydrogel-forming material or hydrogel can be configured for surgical administration. In other words, it can be provided according to the invention that the hydrogel-forming material or hydrogel is surgically used or applied.

In a preferred embodiment, however, the hydrogel-forming material or hydrogel is configured for microinvasive administration. In other words, it is preferred according to the invention if the hydrogel-forming material or hydrogel is microinvasively used or administered.

In a particularly preferred embodiment, the hydrogel-forming material or hydrogel is configured for facet joint infiltration, i.e. infiltration, preferably injection, into the facet joints. In other words, it is preferred according to the invention if the hydrogel-material or hydrogel is used for facet joint infiltration or administered, preferably injected, into the facet joints.

It is particularly preferable if the hydrogel-forming material or hydrogel is configured for intraarticular facet joint infiltration, i.e. for infiltration, preferably injection, into the articular capsules of the facet joints. In other words, it is particularly preferred if the hydrogel-forming material or hydrogel is used for intra-articular facet joint infiltration or administered, preferably injected, into the articular capsules of the facet joints.

Alternatively or in combination, the hydrogel-forming material or hydrogel can be configured for periarticular facet joint infiltration, i.e. for infiltration, preferably injection, into the vicinity of the facet joints or around them (outside of the articular capsules). In other words, the hydrogel-forming material or hydrogel can be alternatively or additionally used for periarticular facet joint infiltration or administered, preferably injected, into the vicinity of the facet joints or around them (outside of the articular capsules).

With respect to further features and advantages of the hydrogel-forming material or hydrogel, in particular of the crosslinkable protein and the crosslinking agent, the above description is incorporated by reference in its entirety in order to avoid repetitions. The statements made in the preceding description, i.e. in the description of the first aspect of the invention, with respect to the crosslinkable protein and the crosslinking agent also apply to the hydrogel material or hydrogel according to the invention.

According to a third aspect, the invention relates to a medical kit, i.e. a kit that can be used in the field of medicine, for use in the prevention (prophylaxis) and/or treatment, in particular symptomatic treatment, of a disease of the facet joints (facet joint disease), in particular in humans and animals, and/or a medical kit for use in the replacement and/or regeneration of articular facets, in particular human or animal articular facets.

The kit comprises the following components, which are spatially separated from each other:

a) a crosslinkable protein and
b) a crosslinking agent.

In general, the kit can be configured for surgical application. In other words, it can be provided according to the invention that the kit is surgically used or applied.

In a preferred embodiment, however, the kit is configured for microinvasive administration. In other words, it is preferred according to the invention if the kit is microinvasively used or applied.

In a particularly preferred embodiment, the kit is configured for facet joint infiltration, i.e. for infiltration, preferably injection, into the facet joints. In other words, it is particularly preferred according to the invention if the kit is used for facet joint infiltration or the kit components a) and b) are administered, preferably injected, into the facet joints.

It is particularly preferred if the kit is configured for intra-articular facet joint infiltration, infiltration, preferably injection, into the articular capsules of the facet joints. In other words, it is particularly preferred if the kit is used for intraarticular facet joint infiltration or the kit components a) and b) are administered, preferably injected, into the articular capsules of the facet joints.

Alternatively or in combination, the kit can be configured for periarticular facet joint infiltration, i.e. infiltration, preferably injection, into the vicinity of the facet joints or around them (outside of the articular capsules). In other words, the kit can alternatively or additionally be used for periarticular facet joint infiltration or the kit components a) and b) can be administered, preferably injected, into the vicinity of the facet joints or around them (outside of the articular capsules).

In an advantageous embodiment, each of the kit components a) and b) is contained in a separate receptacle. In other words, according to an advantageous embodiment, the kit according to the invention is a kit comprising a first receptacle that contains a crosslinkable protein and a second receptacle that contains a crosslinking agent. For example, each of the receptacles may be a cartridge, a container, a syringe receptacle, in particular a syringe cylinder, or a chamber of a multichamber discharge device, in particular a two-chamber discharge device.

In a further embodiment, the kit component a) and/or the kit component b) can further comprise an active ingredient and/or a contrast agent. With respect to a suitable active ingredient and/or contrast agent, the above description is incorporated in its entirety by reference. The statements made in the preceding description with respect to possible active ingredients and contrast also apply to the kit component a) and/or the kit component b).

With respect to further features and advantages of the kit, in particular of the crosslinkable protein and the crosslinking agent, the above description is also incorporated in its entirety by reference in order to avoid repetition. The statements made with respect to the crosslinkable protein and the crosslinking agent in the preceding description, in particular in the description of the first aspect of the invention, also apply to the kit according to the invention.

According to a fourth aspect, the invention relates to a multichamber discharge device, in particular a two-chamber discharge device, for use in the prevention (prophylaxis) and/or in treatment, in particular symptomatic treatment, of disease of the facet joints (facet joint disease), in particular in humans or animals, and/or a multichamber discharge device, in particular a two-chamber discharge device, for use in the replacement and/or regeneration of articular facets, in particular human or animal articular facets.

The multichamber discharge device comprises a first chamber containing a crosslinkable protein and a second chamber containing a crosslinking agent.

In a further embodiment, the multichamber discharge device comprises a mixing device. In the mixing device, the protein and the crosslinking agent are mixed, for example by means of a static mixer, before the mixture is discharged via a suitable discharge opening.

In a further embodiment, the first chamber and/or the second chamber can further comprise an active ingredient and/or a contrast agent. With respect to a suitable ingredient and/or contrast agent, the above description is incorporated in its entirety by reference. The statements made in the preceding description with respect to possible active ingredients and contrast agents also apply to the multichamber discharge device according to the invention.

With respect to further features and advantages of the multichamber discharge device, in particular of the crosslinkable protein and the crosslinking agent, the above description is also incorporated in its entirety by reference in order to avoid repetition. The statements made in the preceding with respect to the crosslinkable protein and the crosslinking agent in the above description, in particular in the description of the first aspect of the invention, also apply to the multichamber discharge device according to the invention.

According to a fifth aspect, the invention relates to a further medical composition, i.e. a composition usable in the field of medicine, for use in the prevention (prophylaxis) and/or treatment, in particular symptomatic treatment, of a disease of the facet joints (facet joint disease), in particular in humans or animals, and/or a further medical composition for use in the replacement and/or regeneration of articular facets, in particular human or animal articular facets.

The further medical composition is characterized in particular by comprising a crosslinking agent.

With respect to further features and advantages of the further medical composition, in particular the crosslinking agent and possible active ingredients and/or contrast agents, the above description is also incorporated in its entirety by reference in order to avoid repetition. The statements made in the preceding description with respect to the above-described medical composition, in particular the crosslinkable protein and possible active ingredients and/or contrast agents, and the crosslinking agent also apply to the further medical composition.

Finally, the invention relates to a method for the prevention (prophylaxis) and/or treatment of a disease of the facet joints and/or the replacement and/or regeneration of articular facets, wherein a crosslinkable protein in combination with a crosslinking agent is administered into the facet joints of a human or animal patient.

In general, the administration of the crosslinkable protein and the crosslinking agent can take place surgically or in a minimally invasive manner.

In a preferred embodiment, however, administration of the crosslinkable protein and the crosslinking agent is carried out by injection into the facet joints.

In a particularly preferred embodiment, administration of the crosslinkable protein and the crosslinking agent takes place by means of intra-articular and/or periarticular, preferably intra-articular, facet joint infiltration.

With respect to further features and advantages of the method, in particular of the crosslinkable protein and the crosslinking agent, the above description is also incorporated in its entirety by reference in order to avoid repetition. The statements made in the preceding description with respect to the crosslinkable protein and the crosslinking agent also apply to the method according to the invention.

Further features and advantages of the invention can be found in the following description of preferred embodiments in the form of examples. Here, individual features can be implemented individually or in combination with one another. The preferred embodiments serve only to further describe and improve understanding of the invention, without being limited thereto.

### EXAMPLE SECTION

1. Production of Maleimide-Functionalized Serum Albumin of Bovine Origin.

250 mg of human, leporid, bovine, or ovine serum albumin (Sigma-Aldrich) was dissolved in 5 ml of 1 M sodium borate (pH 8.2).

Furthermore, 106 mg of 3-maleimidopropionic acid-N-hydroxysuccinimide ester (SMP, Obiter Research, Urbana, Ill., USA) was dissolved in 950 μl of dimethylformamide (DMF). Insoluble material was separated by centrifugation. 500 μl of the supernatant was then added to the albumin solution. After this, incubation was carried out for a further 60 min at room temperature. 500 μl of 3 M sodium acetate (pH 4.7) was then added and dialysis was carried out three times against 1 l of PBS on ice. The dialysate was then concentrated by ultrafiltration (YM-3 membrane, Millipore) to a volume of 3.5 ml, filtration sterilized, and stored at −80° C.

The serum albumin functionalized in this manner can be crosslinked in the presence of hyaluronic acid by adding SH crosslinkers. Particularly suitable in this case is the crosslinker bis-thiol-polyethylene glycol, which bears a thiol group at each terminal. Bis-thio-PEG is commercially available. The crosslinker with a molecular mass of 10,000 g/mol was used. The most favorable gel formation was obtained when the SH groups of bis-thio-PEG and the maleimide groups of the albumin were present in equimolar concentrations.

The hyaluronic-acid-containing gels were then tested with respect to their capacity to release hyaluronic acid. For this purpose, a hydrogel was produced by crosslinking 3 mmol/L of albumin-bound maleimide groups and 3 mmol/L of polyethylene glycol-bound thiol groups that additionally contained 1% (w/v) hyaluronic acid with a total volume of 1 ml of phosphate buffered (pH 7) isotonic saline solution. An area of this hydrogel measuring approx. 2 cm² was layered with a volume of 1 ml of phosphate-buffered isotonic saline solution and incubated at 37° C. The isotonic saline solution was replaced in each case after periods of three days by fresh isotonic saline solutions. The used isotonic saline solution was dialyzed in each case against distilled water and concentrated by lyophilization.

As a reference, the same experimental arrangement was set up, but unfunctionalized polyethylene glycol was used instead of the bisthiofunctionalized crosslinker.

The saline solutions used were analyzed with a gel filtration chromatography unit coupled with a viscosity detector.

It was observed that hyaluronic acid was released from the hydrogel into the overlayered saline solution significantly more slowly than from the uncrosslinked reference material.

1.-18. (canceled)

19. A medical composition for use in the prevention and/or treatment of disease of the facet joints and/or in the replacement and/or regeneration of the articular facets, characterized in that the composition comprises a crosslinkable protein and is convertible by means of a crosslinking agent into a hydrogel-forming material or a hydrogel.

20. The medical composition of claim 19, characterized in that the facet joints are lumbar facet joints.

21. The medical composition of claim 19, characterized in that the disease is a degenerative disease, preferably arthrosis, in particular secondary or symptomatic arthrosis.

22. The medical composition of claim 19, characterized in that the composition is configured for facet joint infiltration, in particular intra-articular facet joint infiltration.

23. The medical composition of claim 19, characterized in that the composition is an acellular composition.

24. The medical composition of claim 19, characterized in that the protein is functionalized by means of at least one group selected from the group comprising maleimide groups, vinyl sulfone groups, acrylate groups, alkyl halide groups, azirine groups, pyridyl groups, thionitrobenzoic acid groups, arylating groups and combinations thereof.

25. The medical composition of claim 19, characterized in that the protein is a maleimide-functionalized protein, in particular a maleimide-functionalized albumin, preferably a maleimide-functionalized serum albumin.

26. The medical composition of claim 19, characterized in that the protein is contained in an amount of 0.3 wt % to 20 wt %, in particular 0.4 wt % to 5 wt %, preferably 0.5 wt % to 2 wt %, based on the total weight of the composition.

27. The medical composition of claim 19, characterized in that the composition further comprises at least one active ingredient selected from the group comprising a chondroprotective agent, an anti-inflammatory, an analgesic, an antibiotic, a local anesthetic, a cytostatic, a differentiation factor, a modulation factor, an immunosuppressant, an immunostimulant active ingredient, an apoptosis-inducing active ingredient and mixtures of two or more of the above-mentioned active ingredients.

28. The medical composition of claim 27, characterized in that the chondroprotective agent is selected from the group comprising hyaluronic acid, chondroitin sulfate, glucosamine sulfate, oxaceprol, vitamin E, ademetionine and mixtures of two or more of the above-mentioned chondroprotective agents.

29. The medical composition of claim 27, characterized in that the chondroprotective agent is contained in an amount of 0.1 wt % to 5 wt %, in particular 0.2 wt % to 2 wt %, preferably 0.3 wt % to 1 wt %, based on the total weight of the composition.

30. The medical composition of claim 27, characterized in that the local anesthetic is selected from the group comprising lidocaine, mepivacaine, prilocaine, articaine, bupivacaine, ropivacaine, etidocaine, dyclonine, procaine, benzocaine, 2-chloroprocaine, oxybuprocaine, tetracaine, fomocaine and mixtures of two or more of the above-mentioned local an-esthetics.

31. The medical composition of claim 27, characterized in that the local anesthetic is contained in an amount of 0.05 wt % to 15 wt %, in particular 0.1 wt % to 10 wt %, preferably 0.2 wt % to 5 wt %, based on the total weight of the composition.

32. The medical composition of claim 27, characterized in that the anti-inflammatory is selected from the group comprising ibuprofen, acetylsalicylic acid, diclofenac, indomethacin, phenylbutazone, dexamethasone, hydrocortisone, prednisolone, prednisone, betamethasone, triamcinolone, essential oils or ex-tracts, particularly from camomile or arnica blossoms, and mixtures of two or more of the above-mentioned antiinflammatories.

33. The medical composition of claim 27, characterized in that the anti-inflammatory is contained in an amount of 0.02 wt % to 15 wt %, in particular 0.05 wt % to 10 wt %, preferably 0.1 wt % to 5 wt %, based on the total weight of the composition.

34. The medical composition of claim 19, characterized in that the composition further comprises at least one crosslinking agent, preferably an SH-modified crosslinking agent, wherein the crosslinking agent is spatially separated from the crosslink-able protein.

35. The medical composition of claim 34, characterized in that the crosslinking agent is selected from the group comprising dithio-polyethylene glycol (dithio-PEG), SH-modified hyaluronic acid, SH-modified dextran, SH-modified polyvinyl alcohol, SH-modified polyvinylpyrrolidone and mixtures of two or more of the above-mentioned crosslinking agents.

36. A medical hydrogel for use in the prevention and/or treatment of a disease of the facet joints and/or in the replacement and/or regeneration of articular facets, characterized in that the hydrogel is obtainable by mixing of a crosslinkable protein and a crosslinking agent.
